(19) United States
(12) Patent Application Publication
(10) Pub. No.: US 2026/0341398 A1
(43) Pub. Date: Oct. 29, 2026

(54) METHODS FOR TARGETING PROTEIN STRUCTURES USING INFORMATION THEORY

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Benjamin D. Greenbaum, New York, NY (US); David Hoyos, New York, NY (US)

(21) Appl. No.: 19/490,314

(22) PCT Filed: Jun. 6, 2024
(86) PCT No.: PCT/US2024/032832

§ 371 Date: Dec. 4, 2025

Related U.S. Application Data
(60) Provisional application No. 63/506,709, filed on Jun. 7, 2023.

(51) Int. Cl. G16B 15/30; G16H 20/10

(52) U.S. Cl. CPC G16B 15/30; G16H 20/10

(57) ABSTRACT

The present disclosure provides methods and systems for selecting therapies for a protein structure based on structural distances among open reading frames (ORFs) encoding diverse proteins using information theory.

### CROSS REFERENCE TO RELATED APPLICATIONS

The present application claims the benefit of and priority to U.S. Provisional Patent Application No. 63/506,709, filed Jun. 7, 2023, which is incorporated herein by reference in its entirety.

### TECHNICAL FIELD

The present disclosure provides methods and systems for selecting therapies for a protein structure based on structural distances among open reading frames (ORFs) encoding diverse proteins via information theory.

### BACKGROUND

A computing system can process input data in a given modality to generate output data of various modalities.

### SUMMARY

Aspects of the present disclosure are related to systems and methods of selecting therapies based on structural distances among proteins encoded by open reading frames (ORFs). One or more processors may obtain, for a subject suffering from or at risk of a pathology, a dataset defining a plurality of protein structures associated with at least one protein. The plurality of protein structures may include a pair of proteins encoded by ORFs. The pair of proteins may include a first protein and a second protein. The one or more processors may identify, for the pair of proteins using the dataset, (i) a first coordinate associated with the first protein and (ii) a second coordinate associated with the second protein, the second coordinate relative to the first coordinate within the plurality of protein structures. The one or more processors may determine an alignment metric between the pair of proteins, using an offset between the first coordinate associated with the first protein and the second coordinate associated with the second protein. The one or more processors may determine a compression metric based on the alignment metric and a reference alignment metric for the pair of proteins without the offset. The one or more processors may generate, based on the compression metric, a distance metric indicating a degree of similarity between the pair of proteins. The one or more processors may select, from a plurality of therapies, a therapy to address the pathology based on the distance metric. The one or more processors may store, using one or more data structures, an association between the subject and the therapy.

In some embodiments, the one or more processors may provide, for presentation, an instruction identifying the therapy to address the pathology in the subject. In some embodiments, the one or more processors may generate (i) a first directional probability distribution and a first angular probability distribution using the first coordinate for the first protein and (ii) a second directional probability distribution and a second angular probability distribution using the second coordinate for the second protein. In some embodiments, the one or more processors may determine (i) a directional alignment metric generated based on the first directional probability distribution for the first protein and the second directional probability distribution for the second protein and (ii) an angular alignment metric based on the first angular probability distribution for the first protein and the second angular probability distribution for the second protein.

In some embodiments, the one or more processors may determine the compression metric as a function of: (i) a directional alignment metric generated based on a first directional probability distribution for the first protein and a second directional probability distribution for the second protein and (ii) an angular alignment metric based on the first angular probability distribution for the first protein and the second angular probability distribution for the second protein. In some embodiments, the one or more processors may determine the directional alignment metric using a reference alignment metric corresponding for the pair of proteins without the offset, in accordance with a predicted local distance difference test (pLDDT).

In some embodiments, the one or more processors may determine that the distance metric does not exceed a threshold. The one or more processors may select the therapy applicable to both the first protein and the second protein of the pair of proteins, responsive to determining that the distance metric does not exceed the threshold. In some embodiments, the one or more processors may determine that the distance metric exceeds a threshold. The one or more processors may select subset of therapies applicable to the first protein and the second protein respectively, responsive to determining that the distance metric exceeds the threshold.

Additionally or alternatively, in some embodiments, the protein structures comprise one or more of retrotransposons, oncogenes, tumor suppressors, receptors, ligands, enzymes, signaling proteins, ribosomal proteins, cytosolic proteins or transcription factors. In some embodiments, the protein structures may include a long-interspersed element-1 (LINE-1) encoding at least one of a ORF1p or ORF2p, TP53 or KRAS.

In some embodiments, the plurality of therapies may include at least one of an anti-cancer drug, an anti-fungal drug, an anti-viral drug, a cardiovascular agent, a CNS agent, an antibacterial drug, an anti-inflammatory drug, or a hormonal drug. In some embodiments, the plurality of therapies comprises at least one of a small molecule, an antibody, a fusion protein, a peptide, or an inhibitory nucleic acid.

In some embodiments, the pathology may include at least one of a cancer, an autoimmune disease, a metabolic disease, cardiovascular disease, a neurodegenerative disease or an infectious disease. In some embodiments, the cancer may include at least one of lung cancer, brain cancer, head and neck cancer, colon cancer, rectal cancer, uterine cancer, endometrial cancer, stomach cancer, ovarian cancer, cervical cancer, pancreatic cancer, bladder cancer, skin cancer, blood cancer, or breast cancer. In some embodiments, the infectious disease may include at least one of a fungal infection, a viral infection or a bacterial infection. In some embodiments, the autoimmune disease may include at least one of anti-neutrophil cytoplasm antibodies (ANCA), ANCA-associated vasculitis (AAV) or giant cell arteritis (GCA) vasculitis, Sjogren's syndrome, inflammatory bowel disease (IBD), Pemphigus vulgaris, lupus nephritis, psoriasis, thyroiditis, Type I Diabetes, Idiopathic thrombocytopenic purpura (ITP), Ankylosing spondylitis, Multiple sclerosis, systemic lupus erythematosus (SLE), rheumatoid arthritis, Crohn's disease, Myasthenia Gravis, neuromyelitis optica (NMO), IgG4-related disease, systemic sclerosis, insulin-dependent diabetes mellitus (IDDM), akylosing spondylitis, atopic dermatitis, uveitis, and Graft-versus-host disease (GVHD). In some embodiments, the neurodegenerative disease may include at least one of Huntington's disease, Parkinson's disease, Alzheimer's disease, ALS, Down's syndrome, dementia pugilistica, cognitive dysfunction syndrome, multiple system atrophy, inclusion body myositosis, hereditary cerebral hemorrhage with amyloidosis of the Dutch type, Nieman-Pick disease type C, cerebral β-amyloid angiopathy, dementia associated with cortical basal degeneration, the amyloidosis of type 2 diabetes, the amyloidosis of chronic inflammation, the amyloidosis of malignancy and Familial Mediterranean Fever, the amyloidosis of multiple myeloma and B-cell dyscrasias, the amyloidosis of prion diseases, Creutzfeldt-Jakob disease, Gerstmann-Straussler syndrome, kuru, scrapie, the amyloidosis associated with carpal tunnel syndrome, senile cardiac amyloidosis, familial amyloidotic polyneuropathy, amyloidosis associated with endocrine tumors. In some embodiments, the metabolic disease may include at least one of adrenoleukodystrophy, diabetes, gaucher disease, glucose galactose malabsorption, hereditary hemochromatosis, lesch-Nyhan syndrome, maple syrup urine disease, Menkes syndrome, Niemann-Pick disease, Obesity, phenylketonuria, Prader-Willi syndrome, Porphyria, Refsum disease, Tangier disease, Tay-Sachs disease, Wilson's disease, and Zellweger syndrome.

### BRIEF DESCRIPTION OF THE DRAWINGS

FIG. 1A Structural Evolutionary Analysis of ORF2p. Structural entropy (structural Shannon entropy) in ORF2p, measured from 57 L1 sequences from diverse vertebrates and plants and smoothed by averaging a 20-residue sliding window is lowest in the ancestral palm domain and highest.

FIG. 1B: Structural entropy correlates highly with retrotransposition (retroT, **** p<0.0001, two tailed t-test), comparing to retroT measurements from 417 consecutive scanning trialanine mutants of ORF2p.

FIG. 1C: Mapping retrotransposition and structural entropy onto the structure of ORF2p highlights the overall concordance, as well as a notable discordance in the helix clamp around residue Y823 (inset).

FIG. 1D: Structural perplexity from ORF2p of a curated set of 50 proteins; structural perplexity is an information theoretic measurement of structural distance between two proteins.

FIG. 1E: Structural perplexity between all proteins in the set, represented using multidimensional scaling such that pairwise Euclidean distances are preserved.

FIG. 1F: shows high concordance between the two multiple alignment strategies (sequence versus structural) in the reverse transcription (RT) domain in fingers-palm-thumb.

FIG. 2: Ability of procedure to distinguish evolutionary structural distance across large ensembles of proteins. Each line represents a domain of life. These are cumulative density functions of the distributions of similarity of proteins from each domain of life to LINE-1 ORF2p. The larger the x-axis value, the stronger the similarity. Therefore, the less steep the curve, the stronger the 3D protein structure similarity to ORF2p. These utilize predictions from AlphaFold2 for each domain of life. This means that ORF2p is most similar to proteins in Repbase (which contains other retrotransposons), followed by viruses, humans (which contain retrotransposons in their genomes), bacteria, and archaea. This present evolutionary analysis reveals both ORF2p's structural Shannon entropy and uncovered the relationships of specific domains to other RNA and DNA dependent polymerases, in apparent examples of convergent evolution.

FIG. 3 depicts a block diagram of a system for selecting therapies based on structural distances among proteins encoded by open reading frames (ORFs), in accordance with an illustrative embodiment.

FIG. 4 depicts a block diagram of a process to extract metrics from protein data to determine distance metrics for selecting therapies in the system, in accordance with an illustrative embodiment.

FIG. 5 depicts a flow diagram of a method of selecting therapies based on structural distances among proteins encoded by open reading frames (ORFs), in accordance with an illustrative embodiment.

FIG. 6 depicts a block diagram of a server system and a client computer system, in accordance with one or more implementations.

### DETAILED DESCRIPTION

Following below are more detailed descriptions of various concepts related to, and embodiments of, systems and methods for selecting therapies based on structural distances among proteins encoded by open reading frames (ORFs). It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Section A describes finding targetable protein structures related open reading frame proteins using information theory.

Section B describes systems and methods for selecting therapies based on structural distances among proteins encoded by open reading frames (ORFs).

Section C describes a network environment and computing environment which may be useful for practicing various computing related embodiments described herein.

### DEFINITIONS

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form, and a second plurality of amino acids are in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

As used herein “disordered regions (DRs)” are defined as entire proteins or regions of proteins that lack a fixed tertiary structure. The definition of “disorder” as used here applies to the protein backbone instead of the residue side-chains. Thus, the backbones of “ordered protein regions” within a set of proteins have the same Ramachandran angles among the different copies of the protein, whereas “disordered protein regions” would have different, often dynamic, Ramachandran angles among the set members. These DRs are divided into two major classes: (1) Extended (i.e., random coil like) and Collapsed (i.e., molten globule like).

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein)), when compared and aligned for maximum correspondence over a comparison window or designated region as measured using a BLAST or BLAST 2.0 sequence comparison algorithms with default parameters described below, or by manual alignment and visual inspection (e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 25 amino acids or nucleotides in length, or 50-100 amino acids or nucleotides in length.

As used herein, the terms “individual”, “patient”, or “subject” can be an individual organism, a vertebrate, a mammal, or a human. In some embodiments, the individual, patient or subject is a human.

As used herein, “metric” refers to an information theory metric, such as Shannon bits (or Shannon entropy) measuring a degree of uncertainty in a probability distribution. The metric may quantify an amount of information to describe the attribute or metric. For example, the metric may be a sum of a logarithm (e.g., log-base 2) of a probability of each potential outcome.

As used herein, “offset” refers to a difference in coordinate or positional alignment between a pair of proteins. The offset may refer to a number of coordinate positions by which one protein in the pair is to be shifted relative to the other protein of the pair to achieve the alignment.

As used herein, an “open reading frame” refers to a portion of a DNA sequence that does not include a stop codon (which functions as a stop signal). A codon is a DNA or RNA sequence of three nucleotides (a trinucleotide) that forms a unit of genomic information encoding a particular amino acid or signaling the termination of protein synthesis (stop codon). In eukaryotic genes with multiple exons, an ORF only applies to spliced mRNAs which lack introns. Alternatively, an ORF refers to a sequence that is divisible by three and is bounded by stop codons. In this context, an ORF represents parts of a gene rather than the entire gene.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, “protein coordinates” refer to data files that list the atoms in a protein structure and their three-dimensional (3D) location in space along with summary information about the structure, sequence, and experiment. These files are available in several formats (PDBx/mmCIF, PDB, XML). The archive also includes data files containing experimental observations that are used to determine these atomic coordinates. At a minimum, protein coordinates specify the positions of each atom in space, typically with X, Y and Z Cartesian coordinates (or Polar coordinates), and the chemical element each atom represents. Each atom in the coordinate section is identified by a sequential number in the entry file, a specific atom name, the name and number of the residue it belongs to, a one-letter code to specify the chain, its x, y, and z coordinates, and an occupancy and temperature factor.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Before describing the disclosed embodiments in detail, it is to be understood that the present disclosure is not limited to particular compositions or biological systems, which can, of course, vary. It is also to be understood that the terminology used herein is for the purpose of describing particular embodiments only and is not intended to be limiting.

The definitions of certain terms as used in this specification are provided below. Unless defined otherwise, all technical and scientific terms used herein generally have the same meaning as commonly understood by one of ordinary skill in the art to which the present technology belongs.

As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

### A. Finding Targetable Protein Structures Related Open Reading Frame Proteins Using Information Theory

A large fraction of eukaryotic genomes consists of mobile elements: sequences that either encode protein machinery to mediate their propagation or co-opt other mobile element proteins to copy themselves. DNA ‘cut and paste’ transposons, like the maize elements discovered by Barbara McClintock, are no longer active in primates. Instead, recent primate evolution is dominated by RNA ‘copy and paste’ retrotransposons, in which RNA intermediates are integrated into the genome by encoded reverse transcriptase (RT) activity. These are divided into two classes: (1) long-terminal repeat (LTR) transposons, also called endogenous retroviruses (ERVs), similar to HIV-1 but no longer thought active in humans, and (2) active Long INterspersed Element-1 (LINE-1, L1) non-LTR transposons.

Previously considered to be ‘junk DNA’, L1 is the only active protein-coding human transposon and is an important endogenous mutagen. L1 encodes two proteins, open reading frame 1 protein (ORF1p), a homotrimeric chaperone likely involved in nuclear entry, and ORF2p, which has endonuclease (EN) and RT activities and three additional domains with unknown functions. L1s and their hosts have been co-evolving for 1-2 billion years in an arms race: the transposon attempts to copy itself in a process called retrotransposition, while the host defends against this mutagenic process. Indeed, despite multi-layered host defenses that recognize the L1 DNA and RNA sequences, proteins, and retrotransposition intermediates, L1s have successfully maintained themselves in host populations over evolutionary time. Each person inherits ~100 polymorphic and fixed active Lis, which is a small subset of the approximately half a million inactive copies and fragments that represent ~17% of the human genome.

Retrotransposition depends on ribonucleoprotein assembly between ORF1p, ORF2p, and the L1 RNA that encodes them, a property termed ‘cis preference’. ORF2p may copy and insert any bound RNA, including cellular mRNA sequences and RNAs transcribed from Short INterspersed Element (SINE) sequences Alu and SVA (SINE/variable number tandem repeat (VNTR)/Alu). Together, the molecular ‘fossil’ record of these sequences comprises ~50% of the genome. New insertions begin with a priming mechanism called Target Primed Reverse Transcription (TPRT) in which an endonuclease nick on the ‘bottom’ DNA strand liberates a 3′-OH used to prime RT and generate an RNA:DNA hybrid intermediate. The best characterized L1 relatives are insect R2 LINE elements and bacterial group II mobile introns, which diverged from the human lineage ~700 million and ~4 billion years ago, respectively. Both recognize and mobilize distinctive DNA and RNA sequences unique to each element and accordingly give limited insight into L1 mechanisms. Accumulating evidence shows that de-repressed L1 elements can contribute to human pathology through at least three distinct mechanisms: (1) DNA damage from insertions, abortive insertions, and aberrant L1 EN activity, (2) perturbation of cellular homeostasis in response to L1 activation, and (3) sterile inflammation mediated by sensing of RT products.

In cancer, mutagenic L1 activity is associated with structural rearrangements and chromosomal instability, and expression of L1 causes a p53-dependent cell cycle arrest that may create positive selective pressure for p53 mutations to drive tumor progression. In autoimmunity, hypomethylated and overexpressed L1 is observed in diseases such as systemic lupus erythematosus (SLE), Sjogren's syndrome (SS), and psoriasis. Moreover, RT inhibitors have shown promising results in preclinical models of cancer, aging, and inflammation in mice; and in clinical studies for colorectal cancer and Aicardi-Goutieres Syndrome (AGS), a rare Mendelian interferonopathy characterized by overproduction or failure to clear L1 intermediates. Thus, targeting L1 has broad therapeutic potential, but limited understanding of ORF2p structure and function has restricted rational inhibitor development and dissection of the underlying pathophysiology.

The LINE-1 (L1) retrotransposon is an ancient genetic parasite that has written almost half of the human genome through a “copy-and-paste” mechanism catalyzed by its multifunctional enzyme ORF2p. ORF2p reverse transcriptase (RT) and endonuclease activities have been implicated in the pathophysiology of cancer, autoimmunity, and aging making ORF2p a potential therapeutic target. However, a lack of structural and mechanistic knowledge has hampered efforts to rationally exploit it. Here, structures of the human ORF2p core by X-ray crystallography and cryo-EM in multiple conformational states are reported. The analyses reveal two novel folded domains, a dynamic closed ring conformation with extensive contacts to RNA template, and associated adaptations that contribute to L1's unique lifecycle and insertion mechanism. This evolutionary analysis reveals both ORF2p's structural Shannon entropy and uncovered the relationships of specific domains to other RNA and DNA dependent polymerases, in apparent examples of convergent evolution. These data provide novel mechanistic insights into L1 polymerization and insertion, shed light on the evolutionary history of L1 elements, and enable rational L1 RT inhibitor development.

To address knowledge gaps in L1 biology and facilitate drug discovery endeavors, systems to purify both full-length and a minimal ‘core’ of ORF2p were established, ORF2p RT activity was studied, and its structure was determined using multiple modalities. The investigation revealed (1) a series of adaptations in the ‘right-handed’ fingers, palm, and thumb RT fold, (2) the presence of two novel domains in the RT core, which is termed ‘tower’ and ‘wrist’, and (3) concerted dynamics of the N-terminal EN and C-terminal domain (CTD). Armed with these structural insights, the evolutionary relationships between conserved structural features in ORF2p are mapped out. The results shed light on previously enigmatic steps in the L1 lifecycle, its roles in pathophysiology, and potential routes to therapeutics.

Structural Insight into L1 Evolution

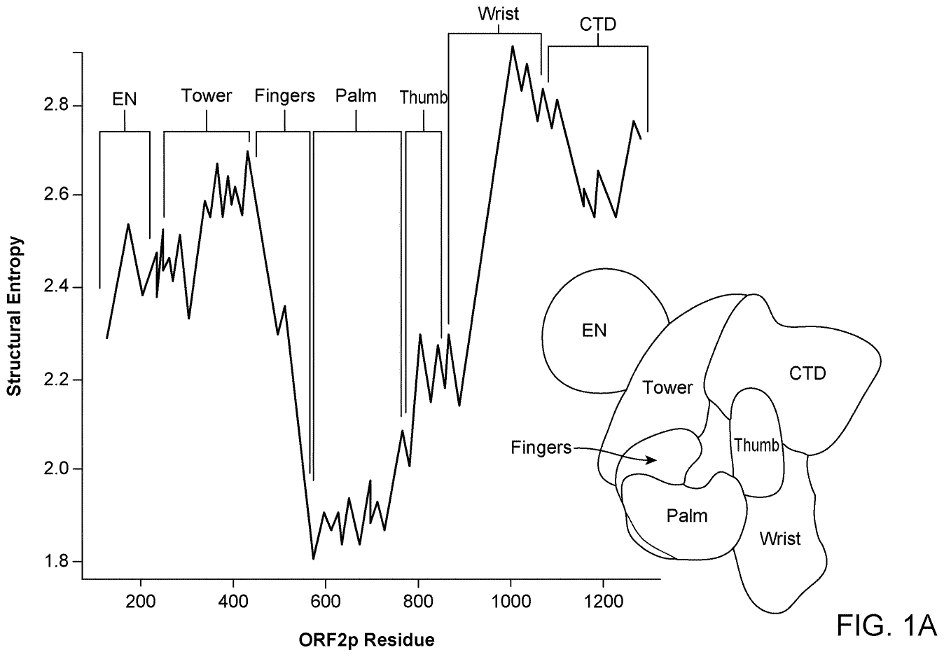

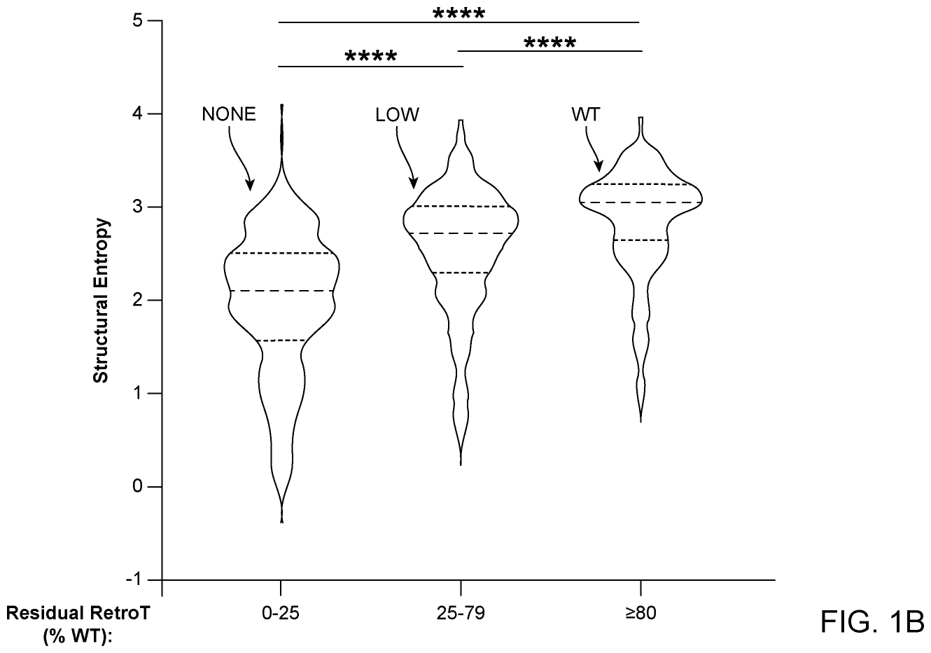

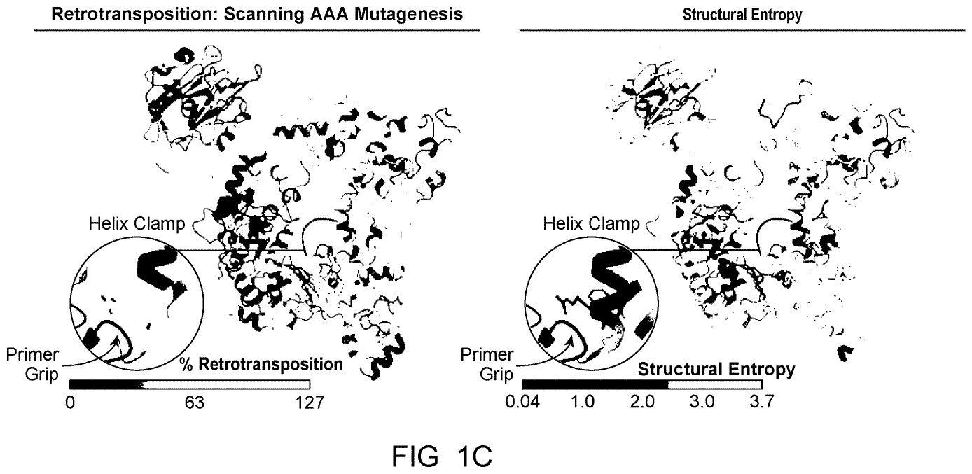

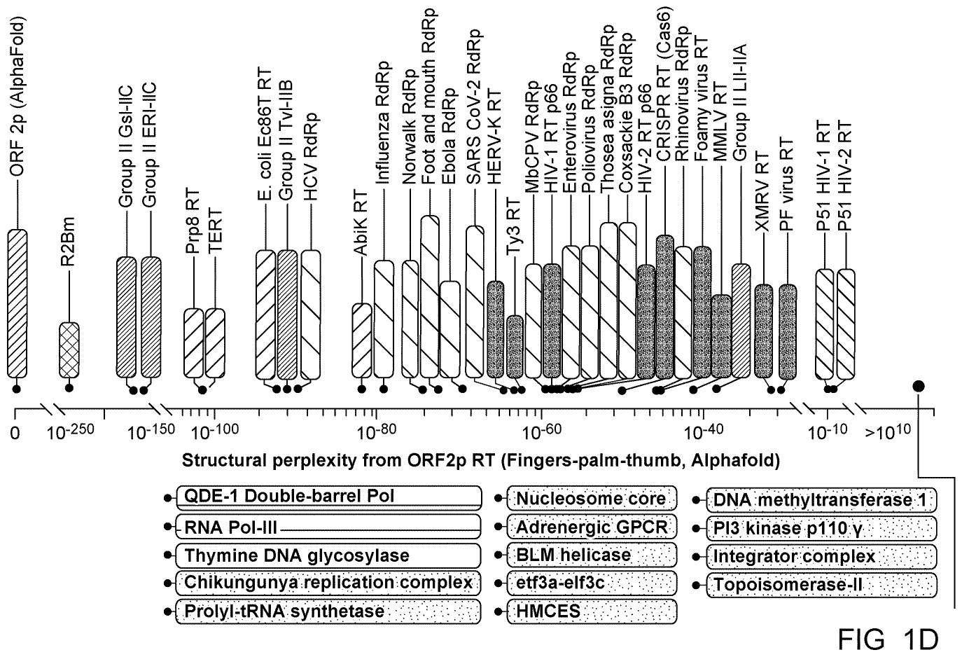

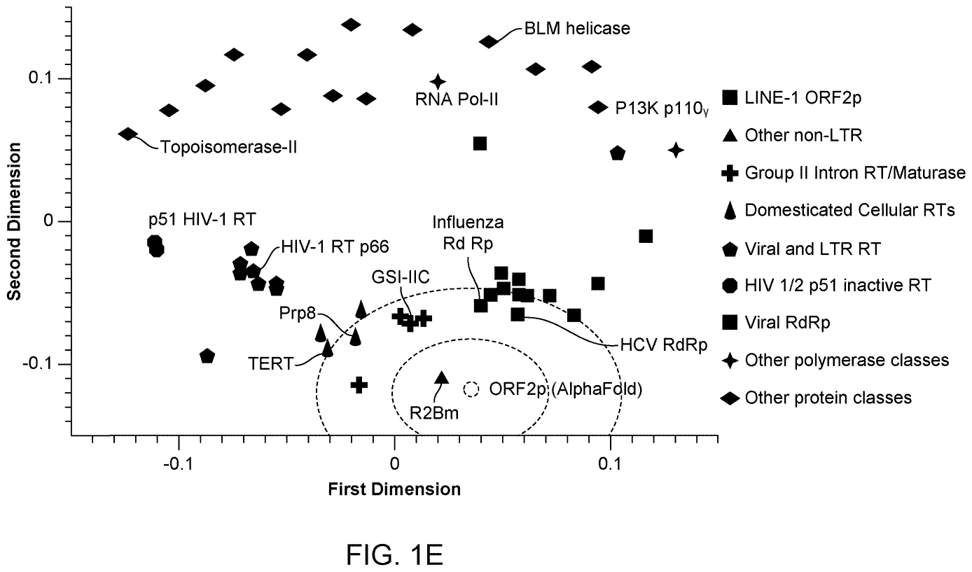

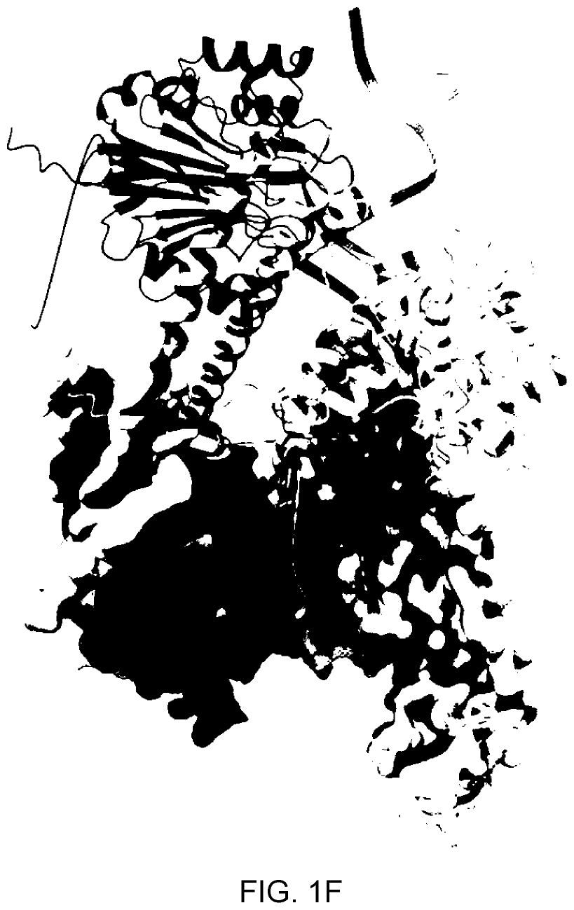

L1 dates to at least the Precambrian era and has no clear evolutionary ancestor amongst viruses. Protein structure was sought to be used to shed light on the conserved features and evolutionary origin of ORF2p that cannot be identified by sequence alignment alone. Multiple sequence/structural alignments and AlphaFold predictions were used to examine conservation of the human ORF2p structure relative to 57 other L1 ORF2p's from vertebrates and plants. By computing and plotting the residue level diversity of the aligned ORF2ps as Shannon entropy (FIG. 1a), high concordance was found between the two multiple alignment strategies (sequence versus structural) in the RT domain (fingers-palm-thumb, FIG. 1f). Despite lower sequence conservation in the tower, wrist, and CTD domains, the structure was conserved, suggesting domain topology is more important than sequence of these domains for L1 function. Leveraging data from a published trialanine mutagenesis library of 417 consecutive AAA ORF2p mutants, in which residual function of mutants was compared to wild-type (WT=100%), structural entropy was found to correlate significantly with residual retrotransposition activity (FIG. 1b,c). As most mutations also resulted in reduced function, these results together suggest that a major evolutionary driving force is optimization of retrotransposition.

Evolutionary relationships between ORF2p and other exogenous and endogenous proteins were next queried, with the intention of identifying shared structural features. First, a set of 50 experimental protein structures was manually curated which represented major families: RTs, RdRps, DdDps, DdDps/RdRps, as well as “controls” which should have little resemblance to the other proteins. The representation of structural similarity was desired in a fashion which would faithfully account for differences in protein length, account for inherent alignment quantity/quality trade-offs, and address a limitation of other methods, such as RMSD, where different relative orientations of otherwise-identical domains results in poor scores.

To do so, an information-theoretic framework was implemented which represented a high-quality alignment as one which reduced the effective information required to transfer the coordinates of two proteins. The structural distance between two proteins is denoted as the “Structural Perplexity”. The smaller this value, the more likely one could guess the coordinates of one structure knowing the coordinates of the other. Plotting structural perplexity from ORF2p for this set (FIG. 1d) shows that it recapitulates close relationships between ORF2p, R2Bm, and group II introns, and that the “control” proteins have extremely high structural perplexities. To better understand relationships between these proteins, the pairwise structural distances were computed across all pairs of proteins and normalized such that the Euclidean distance between pairs of points is proportional to the true structural distances (FIG. 1e).

Across both datasets, proteins in the same functional class typically clustered together in an unsupervised manner, with R2Bm and Group II introns again closest to ORF2p. Domesticated cellular RTs were next closest to ORF2p, followed by viral RdRp's such as HCV and Influenza B, which shared remarkable similarity to ORF2p RT; non-LTR and viral RTs are more distant. Notably, the inactive p51 HIV-1/2 RT subunit was predicted to be far more distant to ORF2p than the active p66 HIV-1/2 RT, despite identical amino acid sequence up to a deletion.

The combined analyses reveal the molecular machine that has written nearly half of the human genome. Understanding L1 structure and function is important both in evolution and, increasingly, in human disease. As summarized above, accumulating evidence shows potentially key contributions of L1 activity, and the host response to it, in common pathologies including cancer, aging, and autoimmunity. The biochemical, structural, and evolutionary analyses show ORF2p is a highly active polymerase that is uniquely adapted for its parasitic lifecycle, with conserved structural features that preserve optimal retrotransposition throughout evolution. Together, these data provide insights into two key underlying mechanisms through which L1 can cause disease: (1) nuclear insertional mutagenesis and related genomic havoc, and (2) cytosolic sensing of the products of reverse transcription.

### Finding Targetable Protein Structures Using Information Theory

Presented herein is a computational algorithm that calculates the structural information distance between two proteins utilizing the information-theoretic concept of “perplexity”. Perplexity is a measure of uncertainty. The perplexity between two proteins estimates the uncertainty of correctly guessing the structure of one protein while knowing the structure of the other protein, given constraints determined by nature. This is a novel, rigorous mathematical description of evolutionary “structural” edits to transition from one structure to another, and thus to transitions in protein function.

This new way of thinking empowers new insights and quantitative predictions into the function and directed design of proteins. Other methods to infer protein function rely on the similarity of one-dimensional amino acid “strings” to infer similar functionality. This approach is highly biased on the proteins that are compared and particularly fails with proteins that are evolutionarily distant. However, protein structure is at least 40% more conserved than sequence. Therefore, a tremendous amount of information is lost with sequence-based approaches.

Furthermore, current methods that align 3D protein structure are inconsistent with each other and often fall in false minima. These methods minimize the Euclidean distance between each pair of proteins' atoms, which makes this a high-dimensional minimization problem which is biased to fail. There exists a compromise between the “quality” of structural similarity and the “quantity” of the number of amino acids being aligned, since the number of degrees of freedom scales with the product of the aligned proteins' lengths. In addition, there is no method that can incorporate uncertainties in the subdomains of protein structures, for example in ordered and disordered protein regions, in a principled manner in these structure comparisons.

The computational algorithm utilizes a rigorous framework in information theory to directly resolve these concerns. The algorithm learns the implicit biophysical and biochemical constraints of three-dimensional structure for a protein. The computational algorithm encodes these principles into an interpretable, computable mathematical language of probabilities. From this, the algorithm can build a vector field map of all likely possible transitions from a protein reference state. Therefore, it can compute the likelihood that a selective process can drive a transition from this reference protein. This is a powerful way of looking at structural similarity. Its foundation in information theory offers the possibility to construct additional mathematical objects describing broad concepts implicated in evolution.

Large-scale sequencing efforts have identified that alterations in our genomes cause cancer, yet each mutation is not the same. Resolving the effect that cancer mutations have on function is a fundamental question underlying our understanding of cancer. This question is made more complicated by the fact that the “universe” of possible mutations is enormous. Therefore, it is imperative to invest in computational approaches that predict the functional consequences of protein mutations. Whereas traditional methods depend on amino acid sequence to infer a mutation's alteration to function, a new function can be appended onto the algorithm which computes a per-residue contribution to the total perplexity and will be a new “oncogenic” score for cancer mutations. This may be possible with mutations in genes that drive cancer such as TPS3, KRAS etc.

Due to the large time and expense associated with testing small-molecule inhibitors in the laboratory and the clinic, in silico screening is an indispensable first step. However, only 14% of pharmaceutical drugs are deemed “successful” after clinical trials. Therefore, there is an urgent need to invest in innovative methods for in silico drug design. In this algorithm, the smaller the perplexity between two proteins the more likely drugs for one protein can work for another protein. In FIG. 1E, Hepatitis C Virus (HCV) is within two standard deviations from LINE-1 ORF2p. This indicates that the principles of drugs against HCV may be applicable to LINE-1 ORF2p, such as sofosbuvir which block viral synthesis.

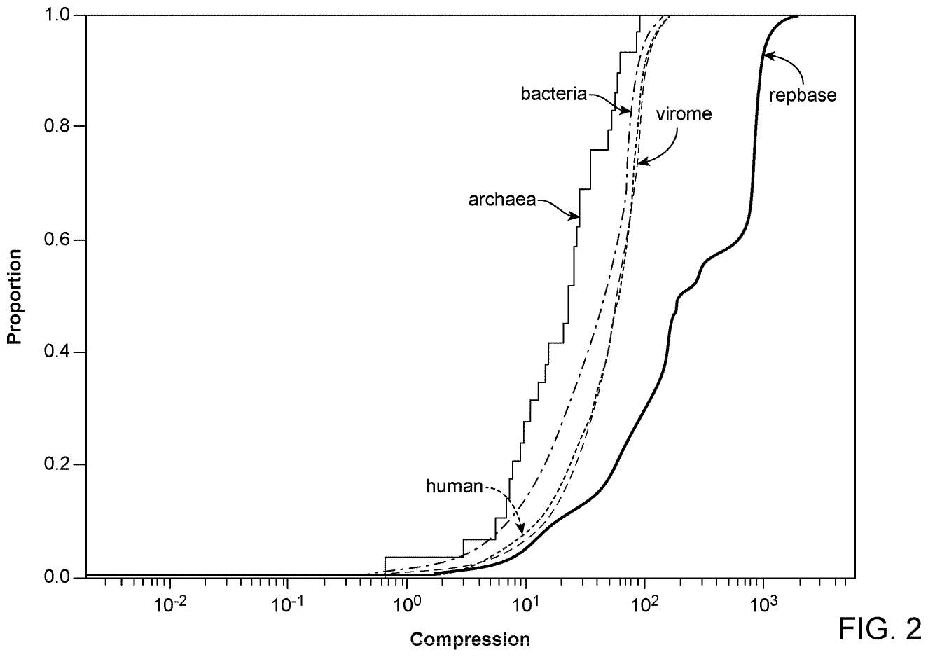

In FIG. 2, each line represents a domain of life. These are cumulative density functions of the distributions of similarity of proteins from each domain of life to the AlphaFold2 prediction of the structure of LINE-1 ORF2p. The larger the x-axis value, the stronger the similarity. Therefore, the less steep the curve, the stronger the 3D protein structure similarity to ORF2p. These utilize predictions from AlphaFold2 for each domain of life. This means that ORF2p is most similar to proteins in Repbase (which contains other retrotransposons), followed by viruses, humans (which contain retrotransposons in their genomes), bacteria, and archaea.

The principal aim is to infer evolutionary similarity via protein structure, as has been done utilizing sequence. There is a fundamental issue with alignments, in that there is a trade-off between the coverage of an alignment and the quality of an alignment. Generally speaking, it may be harder to obtain a high-quality alignment if the region to be aligned is long. On the other hand, it may be easier to find high-quality alignments with short sequences. Additionally, there is inherent uncertainty in protein structure prediction, both in vitro and in silico, particularly with disordered regions (DR) within proteins. This issue is addressed using information theory. Previous efforts have attempted this (MMLigner), although the framework is advanced and rigorously incorporated uncertainty in protein structural alignment for the first-time utilizing concepts from Information Theory. For example, distance metrics which can inform evolutionary similarity in groups of proteins are derived. The method is briefly described below.

### Information Theoretic Treatment of Structural Alignments in R³

Determining what constitutes a good alignment. Suppose there are two sets of protein coordinates (P₁ and P₂). One person (Person 1) wants to communicate both sets of coordinates to another person (Person 2). In one example, Person 1 could send both coordinates separately. The message length (in Shannon bits) could be:

Iₙᵤₗₗ=Iₙᵤₗₗ(P₁)+Iₙᵤₗₗ(P₂)

But if there is similarity, this would be inefficient. A more efficient way is to send the full coordinates of the first protein (P₁), the alignment (A), and any remaining non-aligned coordinates of protein 2 (P₂):

I(A& [symbol] P₁,P₂ [symbol] )=I(A)+I( [symbol] P₁,P₂ [symbol] |A)

This can be shown to be equivalent to:

I(A& [symbol] P₁,P₂ [symbol] )=I(A)+I(P₁|A)+I(P₂|P₁&A)=I(A)+Iₙᵤₗₗ(P₁)+I(P₂|P₁&A)

The efficiency of an alignment can be determined via the compression for a given alignment, C(A):

C(A)=Iₙᵤₗₗ−I(A& [symbol] P₁,P₂ [symbol] )

Here, positive values indicate that the message length with the alignment, I(A& [symbol] P₁, P₂ [symbol] ), is shorter than the message length without the alignment Iₙᵤₗₗ. Negative values indicate the alignment is inefficient, which could result from an alignment with large coverage but poor quality. A value of zero indicates that there is no difference with respect to the message length without the alignment.

Determining the length of a message. The Shannon Information of an event (E) content depends on the probability of its occurrence:

I(E)=−log₂[P(E)]

Encoding Coordinates with a Probability Distribution

### Null Probability Distribution

Protein coordinates need to be encoded with a probability distribution. The null model is encoded in two parts—a radial part and a directional part.

$${I}_{null} ( {\vec{c}}_{i} ) = {I}_{null}^{radius} ( {r}_{i} ) + {I}_{null}^{direction} ( {\hat{x}}_{i} )$$

The radius is encoded with a normal distribution around 3.8 Å with a standard deviation of 0.4 Å.

$${I}_{null}^{radius} ( {r}_{i} ) ∼ - {log}_{2} [ N ( μ = 3.8 , σ = 0.4 ) ]$$

The direction is encoded with a 23-component Kent distribution which parameterizes the angular coordinates of an alpha carbon.

$${I}_{null}^{direction} ∼ - {log}_{2} [ {\sum }_{k = 1}^{\vert M \vert = 23} {w}_{k} {f}_{k} ( \hat{x} ; {\vec{θ}}_{k} ) ]$$

### Probability Distribution Given Alignment

The updated radial and angular probability distributions may depend on the alignment:

$${I}_{align} ( {\vec{c}}_{i} ) = {I}_{align}^{radius} ( {r}_{i} ) + {I}_{align}^{direction} ( {\hat{x}}_{i} )$$

The radial component is transmitted over a x² distribution with three degrees of freedom

$$( {X}_{3}^{2} ) .$$

This constrains the coordinates of protein 2 (P₂) to the coordinates of protein 1 (P₂). However, there is often uncertainty in the coordinates of P₁ and P₂. Therefore, there is a lower probability for these coordinates to be correct and thus a higher contribution to the message length. Notably, in silico predictions of protein structure derived from AlphaFold2 are not drawn from a Boltzmann distribution, which means that it is insufficient to run the same model multiple times to get a sense of the most likely protein structures. The Predicted Aligned Error (PAE) estimates offer an excellent approximation to this error, as the units are in root-mean-square deviation (RMSD) and constitute a distribution across all possible fixed alignments. The standard deviations of these RMSDs for alpha carbons corresponding to indices i and j in P₁ and P₂, respectively, are denoted as

$${σ}_{i}^{{P}_{1}} and {σ}_{i}^{{P}_{2}} ,$$

respectively. Constructing the errors in this way conserves symmetry in the covariance matrices. The contribution to the message length from the radial part is then:

$${I}_{align}^{radius} ( {r}_{i} ) ∼ - {log}_{2} [ {x}_{3}^{2} ( {\sum }_{i}^{\vert A \vert} \frac{{\vert {\vec{r}}_{i}^{{P}_{1}} - {\vec{r}}_{i}^{{P}_{2}} \vert}^{2}}{{σ}_{i}^{{P}_{1}} {σ}_{i}^{{P}_{2}}} ) ]$$

Doing so prioritizes sets of coordinates with low PAE. The directional component is updated as follows. The predicted local distance difference test (pLDDT) is predicted on a per-residue basis for all amino acids. This is expressed in a range [0, 100]. Importantly, these values are proportional to the logarithm of the correct number of rotamers for a given residue, so that:

$${P}_{align}^{direction} ( correct ) ∼ exp [ pLDDT ]$$

Therefore, the contribution to the message length from the directional encoding can be determined from a conditional entropy:

$${I}_{align}^{direction} ( {\hat{x}}_{i} ) ∼ - {log}_{2} [ {P}_{align}^{direction} ( correct ) {P}_{align}^{direction} ( {P}_{2} \vert {P}_{1} , A ) + ( 1 - {P}_{align}^{direction} ( correct ) ) {P}_{null}^{direction} ]$$

Therefore, the efficiency of the compression also depends non-linearly on the quality of the prediction as determined by pLDDT.

### Distance Metric

A distance metric can be defined based on the compression derived from an alignment. The inverse perplexity of the compression is computed as follows:

D=exp[−C(A)]

Therefore, increased compression results in a smaller distance, and negative compression results in larger distances. This offers an advantage over other metrics such as RMSD as this term inherently takes into account the quality and the length of an alignment. Evolutionary trees can be generated using this distance metric in a Bayesian manner. This can be plotted in R² using multidimensional scaling, so that the Euclidean distance is proportional to the true distance in the higher dimensional space.

### Utility of Method

This method is generalizable and is useful for a variety of problems. This method can be used to infer evolutionary relationships of proteins. It can be used to determine erroneously annotated protein sequences based on predicted structural motifs. These efforts are focused on L1 ORF2, but these methods can be applied to other proteins in a straightforward manner. For example, the similarity of structure of diverse cancer-associated mutants can be inferred and efficacy of a drug can be predicted as dependent on local structural similarity. Additionally, the ability to deal with uncertainty in structural predictions may be useful for other forms of uncertainty that arise in the experimental context, such as B-factors. The same principles can be used to define high-quality alignments based on sequence, such as RNA sequences which may form three-dimensional structures.

B. Systems and Methods for Selecting Therapies based on Structural Distances Among Proteins Encoded by Open Reading Frames (ORFs)

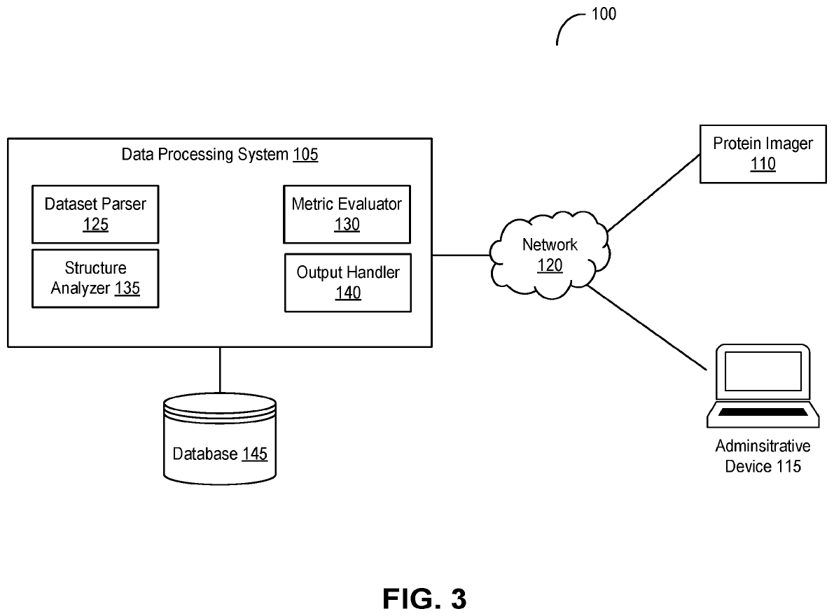

Referring now to FIG. 3, depicted is a block diagram of a system 100 for selecting therapies based on structural distances among proteins encoded by open reading frames (ORFs). In brief overview, the system 100 may include at least one data processing system 105, at least one protein imager 110, and at least one administrative device 115, communicatively coupled with one another via at least one network 120. The data processing system 105 may include at least one dataset parser 125, at least one metric evaluator 130, at least one structure analyzer 135, at least one output handler 140, and at least one database 145, among others. Each of the components in the system 100 as detailed herein may be implemented using hardware (e.g., one or more processors coupled with memory), or a combination of hardware and software as detailed herein in Section C. Each of the components in the system 100 may implement or execute the functionalities detailed herein, such as those described in Section A.

In further detail, the data processing system 105 may (sometimes herein generally referred to as a computing system or a server) be any computing device including one or more processors coupled with memory and software and capable of performing the various processes and tasks described herein. The data processing system 105 can be in communication with the protein imager 110, the administrative device 115, the database 145, and other devices, via the network 120. The data processing system 105 may be situated, located, or otherwise associated with at least one server group. The server group may correspond to a data center, a branch office, or a site at which one or more servers corresponding to the data processing system 105 is situated. In some embodiments, the protein imager 110 may be part of data processing system 105. On the data processing system 105, the dataset parser 125 may acquire a dataset identifying a protein structure from a sample of a subject. The metric evaluator 130 may determine one or more metrics about proteins in the protein structure. The structure analyzer 135 may generate a distance metric measuring structural perplexity of a pair of proteins in the protein structure. The output handler 140 may generate instructions identifying therapy to recommend to the subject based on the distance metric.

The protein imager 110 may be a device or system to generate a protein dataset using a protein taken or obtained from a biological sample. The protein dataset generated by the sequencing device may identify a protein structure by coordinates of individual protein. The protein imager 110 may generate the protein dataset in accordance with any number of imaging techniques, such as X-ray crystallography, nuclear magnetic resonance (NMR) spectroscopy, or cryo-electron microscopy, among others. For example, under X-ray crystallography, the protein imager 110 may scan the crystallized proteins from a biological sample via x-ray exposure, and may perform analysis of the scan from multiple angles to generate the protein dataset identifying the coordinates (e.g., angular and directional coordinates) of the proteins. The protein dataset may be stored and maintained as one or more data files, for example, in accordance with a protein data bank (PDB) format or macromolecular crystallographic information file (mmCIF), among others. In some embodiments, the protein imager 110 may perform protein sequencing, in accordance with any number of sequencing techniques such as Edman degradation, mass spectrometry, or gene sequencing, among others.

The administrative device 115 (sometimes herein referred to as an end user computing device) may be any computing device comprising one or more processors coupled with memory and software and capable of performing the various processes and tasks described herein. The administrative device 115 may be in communication with the data processing system 105 and the protein imager 110 via the network 120. The administrative device 1315 may have at least one display. The administrative device 115 may be associated with an entity (e.g., a clinician) examining the subject, the protein sample, or the biological sample from the subject. The display may present information about the subject provided by the data processing system 105. In some embodiments, the protein imager 110 may be part of the administrative device 115 (e.g., as a computer connected to the scanner).

The database 145 may store and maintain various resources and data associated with the data processing system 105, the protein imager 110, and the administrative device 115, among others. The database 145 may include a database management system (DBMS) to arrange and organize the data maintained thereon. The database 145 may be in communication with the data processing system 105, the protein imager 110, and the administrative device 115, via the network 120. While running various operations, the data processing system 105, the protein imager 110, and the administrative device 115 may access the database 145 to retrieve identified data therefrom. The data processing system 105, the protein imager 110, and the administrative device 115 may also write data onto the database 145 from running such operations.

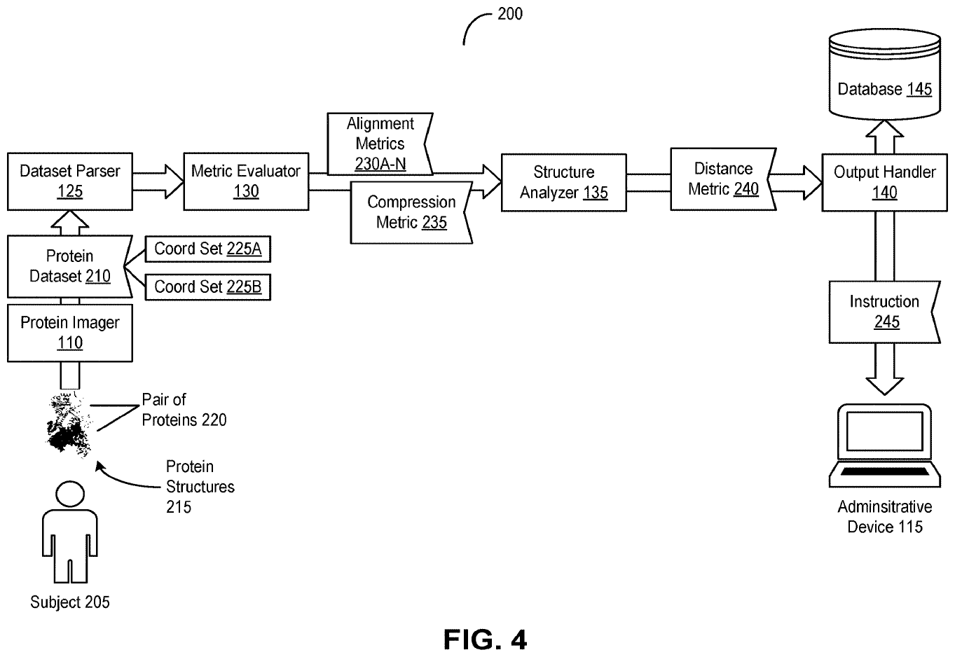

Referring now to FIG. 4 depicts a block diagram of a process 200 to extract metrics from protein data to determine distance metrics for selecting therapies in the system 100. The process 200 may correspond to or include operations in the system 100 to analyze the protein data to select the therapies for at least one subject 205. The subject 205 may be at risk of, suffers from, or is diagnosed with a pathology. The subject 205 may be a human or an animal to be evaluated for a therapy to administer to alleviate, treat, or otherwise address the pathology. A biological sample may be obtained from the subject 205 for analysis. The biological sample may be an organ or a tissue of the subject 205 affected by or associated with the pathology, and may contain proteins useful for analysis.

The pathology may include, for example, a cancer, an autoimmune disease, a metabolic disease, a cardiovascular disease, a neurodegenerative disease or an infectious disease. The cancer may include, for instance, lung cancer, brain cancer, head and neck cancer, colon cancer, rectal cancer, uterine cancer, endometrial cancer, stomach cancer, ovarian cancer, cervical cancer, pancreatic cancer, bladder cancer, skin cancer, blood cancer, or breast cancer, among others. The infectious disease may include, for example, a fungal infection, a viral infection or a bacterial infection, among others. The viral infection may include, for instance, human immunodeficiency virus (HIV), hepatitis (e.g., hepatitis A, B, or C), influenza, cold, varicella virus, herpes simplex, measles, human papillomavirus (HPV), coronavirus, or norovirus, among others. The bacterial infection may include, for example, Streptococcus, Escherichia coli, Proteus, Klebsiella, Mycobacterium tuberculosis, Salmonella, Lyme disease, pertussis, or cellulitis, among others. The autoimmune disease may include, for example, anti-neutrophil cytoplasm antibodies (ANCA), ANCA-associated vasculitis (AAV) or giant cell arteritis (GCA) vasculitis, Sjogren's syndrome, inflammatory bowel disease (IBD), Pemphigus vulgaris, lupus nephritis, psoriasis, thyroiditis, Type I Diabetes, Idiopathic thrombocytopenic purpura (ITP), Ankylosing spondylitis, Multiple sclerosis, systemic lupus erythematosus (SLE), rheumatoid arthritis, Crohn's disease, Myasthenia Gravis, neuromyelitis optica (NMO), IgG4-related disease, systemic sclerosis, insulin-dependent diabetes mellitus (IDDM), akylosing spondylitis, atopic dermatitis, uveitis, or Graft-versus-host disease (GVHD), among others. The neurodegenerative disease may include, for example, Huntington's disease, Parkinson's disease, Alzheimer's disease, ALS, Down's syndrome, dementia pugilistica, cognitive dysfunction syndrome, multiple system atrophy, inclusion body myositosis, hereditary cerebral hemorrhage with amyloidosis of the Dutch type, Nieman-Pick disease type C, cerebral β-amyloid angiopathy, dementia associated with cortical basal degeneration, the amyloidosis of type 2 diabetes, the amyloidosis of chronic inflammation, the amyloidosis of malignancy and Familial Mediterranean Fever, the amyloidosis of multiple myeloma and B-cell dyscrasias, the amyloidosis of prion diseases, Creutzfeldt-Jakob disease, Gerstmann-Straussler syndrome, kuru, scrapie, the amyloidosis associated with carpal tunnel syndrome, senile cardiac amyloidosis, familial amyloidotic polyneuropathy, or amyloidosis associated with endocrine tumors, among others. The metabolic disease may include, for example, adrenoleukodystrophy, diabetes, gaucher disease, glucose galactose malabsorption, hereditary hemochromatosis, lesch-Nyhan syndrome, maple syrup urine disease, Menkes syndrome, Niemann-Pick disease, Obesity, phenylketonuria, Prader-Willi syndrome, Porphyria, Refsum disease, Tangier disease, Tay-Sachs disease, Wilson's disease, or Zellweger syndrome, among others.

The protein imager 110 may produce, output, or otherwise generate at least one protein dataset 210 for at least one subject 205. In generating, the protein imager 110 may generate the protein dataset 210 using at least one biological sample (e.g., tissue or protein sample) from the subject 205. The biological sample may include a set of protein structures 215. The generation of the protein dataset 210 by the protein imager 110 may be in accordance with imaging techniques, such as X-ray crystallography, nuclear magnetic resonance (NMR) spectroscopy, or cryo-electron microscopy. For example, under X-ray crystallography, proteins including the protein structures 215 may be taken from a sample of the subject 205, then crystallized, and then exposed with x-ray scans. The protein image r110 may acquire or scan the x-rays diffracted from the crystalized proteins to generate the protein dataset 210. Using the x-ray diffractions, the protein imager 110 may write, construct, or otherwise generate data element to include into the protein dataset 210.

The protein dataset 210 generated by the protein imager 110 may identify or define the set of protein structures 215 associated with at least one protein for the subject 205. ORFs may correspond to an encoded portion of proteins in deoxyribonucleic acid (DNA) or ribonucleic acid (RNA) sequence. The set of protein structures 215 may contain, have, or otherwise include at least one pair of proteins 220 encoded by open reading frames (ORFs). The set of protein structures comprise one or more of retrotransposons, oncogenes, tumor suppressors, receptors, ligands, enzymes, signaling proteins, ribosomal proteins, cytosolic proteins or transcription factors. In some embodiments, the protein structures may include a long-interspersed element-1 (LINE-1) encoding at least one of a ORF1p or ORF2p, TP53 or KRAS, among others. The pair of proteins 220 may include at least one first protein and at least one second protein. The second protein may be positioned, located, or otherwise arranged at an offset within the set of protein structures 215. The offset may be introduced by the encoding by the ORFs within the set of protein structures 215. The pair of proteins 220 may correspond to at least a portion of the data in the protein dataset 210. By extension, the first protein and the second protein may each correspond to a respective portion of the data of the protein dataset 210.

The protein dataset 210 may identify or include at least one set of protein coordinate for each protein in the set of protein structures. The protein dataset 210 may include a first protein coordinate sets 225A for the first protein and a second protein coordinate sets 225B for the second protein in the pair of proteins 220. A protein coordinate set 225A or 225B (referred herein generally as coordinate sets 215) for a given protein may identify or define the corresponding protein in terms of a coordinate system. The coordinate system may be a Cartesian coordinate system or polar coordinate system. When under the polar coordinate system, each coordinate set 225 may include, for example, an angular component and a directional component (sometimes here referred to as a radial component), among others. The angular component may correspond to an angle between a reference direction and a direction of the corresponding protein. The directional component may correspond to a distance between the protein and a fixed origin for the coordinate system. When under the Cartesian coordinate system, the coordinate set 225 may include an x-component, a y-component, and a z-component, among others. With the generation, the protein imager 110 may send, transmit, or otherwise provide the protein dataset 210 to the data processing system 105.

The dataset parser 125 on the data processing system 105 may receive, identify, or otherwise obtain the protein dataset 210 for the subject 205 from the protein imager 110. Upon obtaining, the dataset parser 125 may parse or process the protein dataset 210 to extract or identify the set of protein structures 215 defined in the protein dataset 210. From the definition of the set of protein structures 215, the data parser 125 may extract or identify at least one pair of proteins 215 encoded by ORFs using the corresponding portions of the protein dataset 210. For the pair of proteins 220 within the set of protein structure 215, the data parser 125 may determine or identify the coordinate set 225A associated with the first protein and the coordinate set 225B for the second protein. The coordinate set 225B may be relative to the first coordinate set 225A within the set of protein structures 215 (e.g., due to the encoding by ORFs). From the coordinate set 225, the data parser 125 may determine or identify the individual components, such as the angular component and directional component.

For each coordinate set 225, the dataset parser 125 may encode, determine, or otherwise generate at least one probability distribution. The probability distribution may include, for example, a Kent distribution (e.g., 23-component Kent distribution), normal (or Gaussian) distribution, Cauchy distribution, Dirichlet distribution, Poisson distribution, or Laplace distribution, among others. The probability distribution may be added to the coordinate sets 225 to reflect or capture uncertainty of the components of the coordinate set 225 for the corresponding protein. In some embodiments, the dataset parser 125 may generate a corresponding probability distribution for each component of the coordinate set 225 using the value for the component. For example, the dataset parser 125 may generate a probability distribution for an angular component and another probability distribution for a directional component of the coordinate set 225A for the first protein of the pair of proteins 220. The dataset parser 125 may generate a probability distribution for an angular component and another probability distribution for a directional component of the coordinate set 225B for the second protein of the pair of proteins 220. The median or centroid point for the probability distribution may be set to the value of component. For example, the probability distribution encoding the first coordinate set 225A may have a mean at the value for the angular and direction component values.

The metric evaluator 130 on the data processing system 105 may calculate, generate, or otherwise determine at least one alignment metric 230A-N (hereinafter generally referred to as alignment metrics 230) between the pair of proteins 220 in the set of protein structures 215. The alignment metric 230 may be determined using at least one offset between the coordinate set 225A for the first protein and the coordinate set 225B for the second protein in the pair of proteins 220. The alignment metric 230 may be defined in terms of information theory metrics, such as Shannon entropy (or Shannon bits). The offset may correspond to a difference in alignment between the first protein and the second protein of the pair of proteins 220 in the set of protein structures 215. In some embodiments, the metric evaluator 130 may determine a set of alignment metrics 230 corresponding to the components (e.g., angular and directional components) of the coordinate system for the coordinate set 225.

For each component, the metric evaluator 130 may determine the alignment metric 230 based on the probability distributions of the corresponding components of the coordinate sets 225 for the pair of proteins 220. In determining, the metric evaluator 130 may calculate, generate, or otherwise determine an offset metric corresponding to the difference in alignment, using the probability distribution for the coordinate sets 225A and 225B. The offset metric may be determined in accordance with an approximate or error function, such as a predicted aligned error (PAE), root-mean sequence deviation (RMSD), or cross-entropy error, among others. The metric evaluator 130 may determine the offset between the pair of proteins 220 in the set of structures 215 using the protein dataset 210 in accordance with a protein alignment algorithm (e.g., Needleman-Wunsch algorithm, distance-matrix alignment (DALI), template modeling alignment, or Multiple protein structural alignment algorithm (MUSTANG)). The offset metric may be determined for each components of the coordinate sets 225.

The metric evaluator 130 may determine the alignment metric 230 for the component as a function of the probability distributions of the corresponding component and the offset metric also for the same component. In some embodiments, the function may be based on properties of the probability distributions, such as mean, expected value, standard deviation, or other statistical characteristics, among others. The function may be different along each component. For instance, the function for the radial component may be:

$${I}_{align}^{radius} ( {r}_{i} ) ∼ - {log}_{2} [ {x}_{3}^{2} ( {\sum }_{i}^{\vert A \vert} \frac{{\vert {\vec{r}}_{i}^{{P}_{1}} - {\vec{r}}_{i}^{{P}_{2}} \vert}^{2}}{{σ}_{i}^{{P}_{1}} {σ}_{i}^{{P}_{2}}} ) ]$$

where standard deviations of these RMSDs for alpha carbons corresponding to indices i and j in P₁ and P₂, respectively, are denoted as

$${σ}_{i}^{{P}_{1}} and {σ}_{i}^{{P}_{2}} ,$$

respectively.

In some embodiments, the metric evaluator 130 may calculate, generate, or otherwise determine at least one reference alignment metric for the pair of proteins 220 without the offset. The reference alignment metric may correspond to the alignment metric, factoring out the offset introduced by the ORF encoding in the pair of proteins 220 of the set of protein structures 215. For each component, the metric evaluator 130 may determine the reference alignment metric as a function of the coordinate sets 225A and 225B for the pair of proteins 220 without the offset. The function may be, for example, predicted local distance difference test (pLDDT). The function may also depend on the component. In some embodiments, the metric evaluator 130 may determine the alignment metric 230 for at least one component, using the reference alignment metric for the same component. The alignment metric 230 for the component may be determined in accordance with the function for the component. The function for the directional component may be, for instance:

$${I}_{align}^{direction} ( {\hat{x}}_{i} ) ∼ - {log}_{2} [ {P}_{align}^{direction} ( correct ) {P}_{align}^{direction} ( {P}_{2} \vert {P}_{1} , A ) + ( 1 - {P}_{align}^{direction} ( correct ) ) {P}_{null}^{direction} ]$$

The metric evaluator 130 may calculate, generate, or otherwise determine at least one compression metric 235 based on the alignment metrics 230 and at least one reference alignment metric for the pair of proteins 220 without any offset. The compression metric 235 may identify or indicate a difference between the alignment metric 230 and the reference alignment to compare the pair of proteins 220 with the offset or without any offset. The compression metric 235 may also be defined in terms of information theory metric, such as Shannon entropy (or Shannon bits). In some embodiments, the metric evaluator 130 may determine the compression metric 235 as a function of the set of alignment metrics 230 corresponding to the set of components for the coordinate set 225. The function may be of, for example, the alignment metric 230 for the directional component and the alignment metric 230 for the angular component. The function may include, for example, a summation or a weighted average of the alignment metrics 230.

The structure analyzer 135 on the data processing system 105 may calculate, determine, or otherwise generate at least one distance metric 240 based on the compression metric 235. The distance metric 240 may identify, define, or otherwise indicate a degree of similarity between the first protein and the second protein in the pair of proteins 220. In some embodiments, the structure analyzer 135 may generate the distance metric 240 as a function of the compression metric 235. The function may have an inverse relationship with the value of the compression metric 235. In general, the greater the compression metric 235, the smaller the distance metric 240 may be, which may be correlated with higher similarity between the pair of proteins 220. Conversely, the lower the compression metric 235, the higher the distance metric 240 may be, which may be correlated with lower similarity between the pair of proteins 220 in the set of protein structures 215 in the subject 205.

Based on the distance metric 240, the output handler 140 on the data processing system 105 may identify or select at least one therapy from a set of candidate therapies to address the pathology of the subject 205. The set of candidate therapies may include, for example, an anti-cancer drug, an anti-fungal drug, an anti-viral drug, a cardiovascular agent, a CNS agent, an antibacterial drug, an anti-inflammatory drug, or a hormonal drug, among others. The anti-cancer drug may include, for instance, the anti-cancer drug comprises at least one of an immune checkpoint blockade therapy (e.g., an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, or an anti-LAG-3 antibody), alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, T cells, bisphosphonate therapy agents and targeted biological therapy agents (e.g., therapeutic peptides described in U.S. Pat. No. 6,306,832, WO 2012007137, WO 2005000889, WO 2010096603 etc.). In some embodiments, the at least one additional therapeutic agent is a chemotherapeutic agent. Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), bevacizumab, oxaliplatin, melphalan, etoposide, mechlorethamine, bleomycin, microtubule poisons, annonaceous acetogenins, or combinations thereof. In some embodiments, the candidate therapy comprises a small molecule, an antibody, a fusion protein, a peptide, or an inhibitory nucleic acid (e.g., sgRNA, siRNA, aptamer etc.), among others.

To select, the output handler 140 may compare the distance metric 240 with a threshold. The threshold may delineate, identify, or otherwise define a value for the distance metric 240 at which to select a given candidate therapy to address the pathology of the subject 205. In general, the smaller the distance metric 240, the more likely the therapy may be at affecting both proteins in the pair of proteins 220. Conversely, the higher the distance metric 240, the less likely the therapy may be at affecting both proteins in the pair of proteins 220. If the distance metric 240 exceeds the threshold, the output handler 140 may select a first therapy to applicable to the first protein and a second therapy applicable to the second protein in the pair of proteins 220. Therapies affecting one protein of the pair of proteins may include, for example, an anti-cancer drug, anti-fungal drug, anti-viral drug, a cardiovascular agent, a CNS agent, an antibacterial drug, an anti-inflammatory drug, a hormonal drug etc. Otherwise, if the distance metric 240 does not exceed threshold the output handler 140 may select at least one therapy to applicable to both the first protein and the second protein in the pair of proteins 220. Therapies affecting both proteins of the pair of proteins may include, for example, an anti-cancer drug, anti-fungal drug, anti-viral drug, a cardiovascular agent, a CNS agent, an antibacterial drug, an anti-inflammatory drug, a hormonal drug, etc.

With the selection, the output handler 140 may store and maintain an association between the subject 205 (e.g., using an anonymized identifier) and the selected therapy, using one or more data structures on the database 145. The data structure may include, for example, a table, a matrix, a linked list, a binary tree, a heap, a hash table, a queue, or a stack, among others. The association may be between the subject 205, with one or more of the protein dataset 210, the coordinate sets 225, the alignment metrics 230, the compression metric 235, and the distance metric 240, among others. In some embodiments, the output handler 140 produce, create, or otherwise generate at least one instruction 245. The instruction 245 may indicate or identify the therapies to be administered to the subject 205 for addressing the pathology. The instruction 245 may also include information about the subject 205, including the association. With the generation, the output handler 140 may send, transmit, or otherwise provide the instruction 245 to the administrative device 115.

Upon the instruction 245, the administrative device 115 may display, render, or otherwise present the identification of the therapies. The administrative device 115 may also present the information included in the instruction 245. For instance, the administrative device 115 may display the information via a graphical user interface of an application that is interfacing with the data processing system 105. Using the information, a clinician examining the subject 205 may make further determination as to which actions to take or which therapy to administer to the subject 205 to alleviate or treat the pathology.

In this manner, the data processing system 105 may calculate various metrics using information theory metrics regarding the structure of the protein structures from the subject 205 to determine what the best therapy is to apply in view of the structural similarities. For example, the data processing system 105 may encode probability distributions about the coordinate sets 225 for each protein in the pair of proteins 220 to account for uncertain variability as to the actual position of the protein within the set of protein structures 215. With the encoding, the data processing system 105 may use the probability distributions as well as uncertainty of the offset introduced by ORF encoding in the pair of proteins 220, to derive information theory metrics. The information theory metrics may correspond to a length of bits that would be entailed in communicating the coordinates accounting for the offset, and may account for the uncertainty introduced from the offset. This may be in contrast to sequencing-based techniques, that are much more computationally complex thus consume more computing resources (e.g., in terms of processor and memory). Based on the information theory-based metrics, the data processing system 105 may determine the structural similarity of the pair of proteins 220, and may select the candidate therapy that would be the most effective given the structural similarity of the proteins as well as the pathology affecting the subject 205. This approach may thus improve clinical outcomes for the subject 205 at addressing the symptoms related to the pathology or its underlying causes.

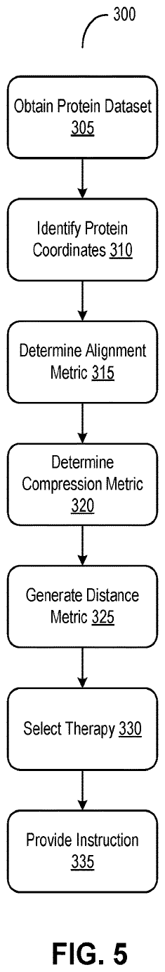

Referring now to FIG. 5 depicts a flow diagram of a method 300 of selecting therapies based on structural distances among proteins encoded by open reading frames (ORFs). The method 300 can be implemented by any components detailed herein, such as the system 100 or the system 400. Under the method 300, a computing system may obtain a protein dataset (305). The computing system may identify protein coordinates of pair of proteins in from the protein dataset (310). The computing system may determine an alignment metric between the pair of proteins (315). The computing system may determine a compression metric using the alignment metric (320). The computing system may generate a distance metric based on the compression metric (325). The computing system may select a therapy from a set of candidate therapies using the distance metric (330). The computing system may provide an instruction to identify the selected therapy (335).

### C. Computing and Network Environment

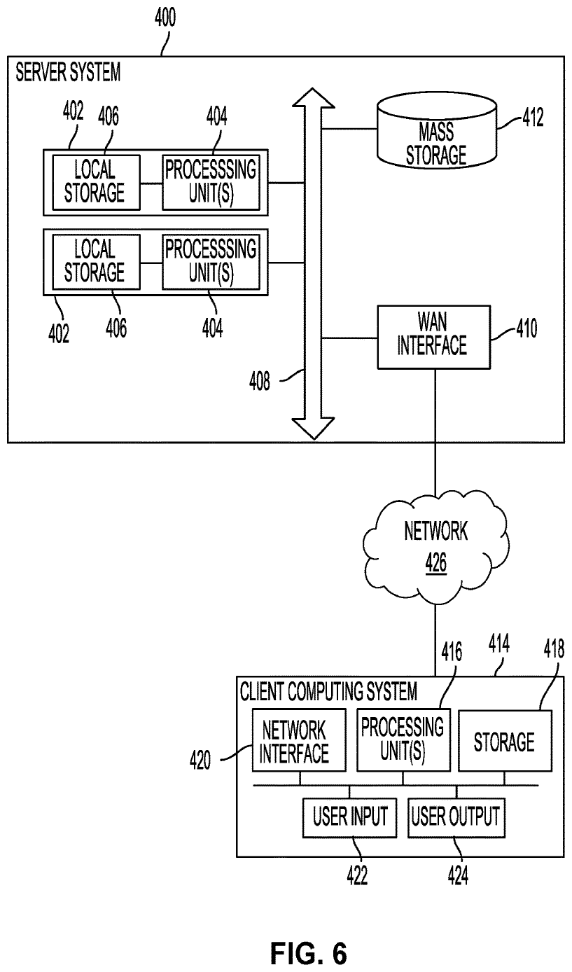

Various operations described herein can be implemented on computer systems. FIG. 6 shows a simplified block diagram of a representative server system 400, client computing system 414, and network 426 usable to implement certain embodiments of the present disclosure. In various embodiments, server system 400 or similar systems can implement services or servers described herein or portions thereof. Client computing system 414 or similar systems can implement clients described herein. The system 400 described herein can be similar to the server system 400. Server system 400 can have a modular design that incorporates a number of modules 402 (e.g., blades in a blade server embodiment); while two modules 402 are shown, any number can be provided. Each module 402 can include processing unit(s) 404 and local storage 406.

Processing unit(s) 404 can include a single processor, which can have one or more cores, or multiple processors. In some embodiments, processing unit(s) 404 can include a general-purpose primary processor as well as one or more special-purpose co-processors, such as graphics processors, digital signal processors, or the like. In some embodiments, some or all processing units 404 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 404 can execute instructions stored in local storage 406. Any type of processors in any combination can be included in processing unit(s) 404.

Local storage 406 can include volatile storage media (e.g., DRAM, SRAM, SDRAM, or the like) and/or non-volatile storage media (e.g., magnetic or optical disk, flash memory, or the like). Storage media incorporated in local storage 406 can be fixed, removable, or upgradeable as desired. Local storage 406 can be physically or logically divided into various subunits, such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 404 need at runtime. The ROM can store static data and instructions that are needed by processing unit(s) 404. The permanent storage device can be a non-volatile read-and-write memory device that can store instructions and data even when module 402 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 406 can store one or more software programs to be executed by processing unit(s) 404, such as an operating system and/or programs implementing various server functions, such as functions of the system 500 of FIG. 5 or any other system described herein, or any other server(s) associated with system 500 or any other system described herein.

“Software” refers generally to sequences of instructions that, when executed by processing unit(s) 404 cause server system 400 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 404. Software can be implemented as a single program or as a collection of separate programs or program modules that interact as desired. From local storage 406 (or non-local storage described below), processing unit(s) 404 can retrieve program instructions to execute and data to process in order to execute various operations described above.

In some server systems 400, multiple modules 402 can be interconnected via a bus or other interconnect 408, forming a local area network that supports communication between modules 402 and other components of server system 400. Interconnect 408 can be implemented using various technologies, including server racks, hubs, routers, etc.

A wide area network (WAN) interface 410 can provide data communication capability between the local area network (interconnect 408) and the network 426, such as the Internet. Technologies can be used, including wired (e.g., Ethernet, IEEE 802.3 standards) and/or wireless technologies (e.g., Wi-Fi, IEEE 802.24 standards).

In some embodiments, local storage 406 is intended to provide working memory for processing unit(s) 404, providing fast access to programs and/or data to be processed while reducing traffic on interconnect 408. Storage for larger quantities of data can be provided on the local area network by one or more mass storage subsystems 412 that can be connected to interconnect 408. Mass storage subsystem 412 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 412. In some embodiments, additional data storage resources may be accessible via WAN interface 410 (potentially with increased latency).

Server system 400 can operate in response to requests received via WAN interface 410. For example, one of modules 402 can implement a supervisory function and assign discrete tasks to other modules 402 in response to received requests. Work allocation techniques can be used. As requests are processed, results can be returned to the requester via WAN interface 410. Such operations can generally be automated. Further, in some embodiments, WAN interface 410 can connect multiple server systems 400 to each other, providing scalable systems capable of managing high volumes of activity. Other techniques for managing server systems and server farms (collections of server systems that cooperate) can be used, including dynamic resource allocation and reallocation.

Server system 400 can interact with various user-owned or user-operated devices via a wide-area network such as the Internet. An example of a user-operated device is shown in FIG. 4 as client computing system 414. Client computing system 414 can be implemented, for example, as a consumer device such as a smartphone, other mobile phone, tablet computer, wearable computing device (e.g., smart watch, eyeglasses), desktop computer, laptop computer, and so on.

For example, client computing system 414 can communicate via WAN interface 410. Client computing system 414 can include computer components such as processing unit(s) 416, storage device 418, network interface 420, user input device 422, and user output device 424. Client computing system 414 can be a computing device implemented in a variety of form factors, such as a desktop computer, laptop computer, tablet computer, smartphone, other mobile computing device, wearable computing device, or the like.

Processing unit(s) 416 and storage device 418 can be similar to processing unit(s) 404 and local storage 406, as described above. Suitable devices can be selected based on the demands to be placed on client computing system 414; for example, client computing system 414 can be implemented as a “thin” client with limited processing capability or as a high-powered computing device. Client computing system 414 can be provisioned with program code executable by processing unit(s) 416 to enable various interactions with server system 400.

Network interface 420 can provide a connection to the network 426, such as a wide area network (e.g., the Internet), to which WAN interface 410 of server system 400 is also connected. In various embodiments, network interface 420 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 422 can include any device (or devices) via which a user can provide signals to client computing system 414; client computing system 414 can interpret the signals as indicative of particular user requests or information. In various embodiments, user input device 422 can include any or all of a keyboard, touch pad, touch screen, mouse or other pointing device, scroll wheel, click wheel, dial, button, switch, keypad, microphone, and so on.

User output device 424 can include any device via which client computing system 414 can provide information to a user. For example, user output device 424 can include a display to present images generated by or delivered to client computing system 414. The display can incorporate various image generation technologies, e.g., a liquid crystal display (LCD), a light-emitting diode (LED) including organic light-emitting diodes (OLED), a projection system, a cathode ray tube (CRT), or the like, together with supporting electronics (e.g., digital-to-analog or analog-to-digital converters, signal processors, or the like). Some embodiments can include a device, such as a touchscreen, that function as both input and output device. In some embodiments, other user output devices 424 can be provided in addition to or instead of a display. Examples include indicator lights, speakers, tactile “display” devices, printers, and so on.

Some embodiments include electronic components, such as microprocessors, storage and memory that store computer program instructions in a computer-readable storage medium. Many of the features described in this specification can be implemented as processes that are specified as a set of program instructions encoded on a computer-readable storage medium. When these program instructions are executed by one or more processing units, they cause the processing unit(s) to perform various operation indicated in the program instructions. Examples of program instructions or computer code include machine code, such as that produced by a compiler, and files including higher-level code that are executed by a computer, an electronic component, or a microprocessor using an interpreter. Through suitable programming, processing unit(s) 404 and 416 can provide various functionality for server system 400 and client computing system 414, including any of the functionality described herein as being performed by a server or client, or other functionality.

It will be appreciated that server system 400 and client computing system 414 are illustrative and that variations and modifications are possible. Computer systems used in connection with embodiments of the present disclosure can have other capabilities not specifically described here. Further, while server system 400 and client computing system 414 are described with reference to particular blocks, it is to be understood that these blocks are defined for convenience of description and are not intended to imply a particular physical arrangement of component parts. For instance, different blocks can be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatuses, including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies, including but not limited to the specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or by any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer-readable storage media; suitable media include magnetic disk or tape, optical storage media such as compact disk (CD) or DVD (digital versatile disk), flash memory, and other non-transitory media. Computer-readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

1. A method of selecting therapies based on structural distances among proteins encoded by open reading frames (ORFs), comprising:
obtaining, by one or more processors, for a subject suffering from or at risk of a pathology, a dataset defining a plurality of protein structures associated with at least one protein, the plurality of protein structures including a pair of proteins encoded by ORFs, the pair of proteins including a first protein and a second protein;
identifying, by the one or more processors, for the pair of proteins using the dataset, (i) a first coordinate associated with the first protein and (ii) a second coordinate associated with the second protein, the second coordinate relative to the first coordinate within the plurality of protein structures;
determining, by the one or more processors, an alignment metric between the pair of proteins, using an offset between the first coordinate associated with the first protein and the second coordinate associated with the second protein;
determining, by the one or more processors, a compression metric based on the alignment metric and a reference alignment metric for the pair of proteins without the offset;
generating, by the one or more processors, based on the compression metric, a distance metric indicating a degree of similarity between the pair of proteins;
selecting, by the one or more processors, from a plurality of therapies, a therapy to address the pathology based on the distance metric; and
storing, by the one or more processors, using one or more data structures, an association between the subject and the therapy.

2. The method of claim 1, further comprising providing, by the one or more processors, for presentation, an instruction identifying the therapy to address the pathology in the subject.

3. The method of claim 1, further comprising generating, by the one or more processors, (i) a first directional probability distribution and a first angular probability distribution using the first coordinate for the first protein and (ii) a second directional probability distribution and a second angular probability distribution using the second coordinate for the second protein, and
wherein determining the alignment metric further comprises determining (i) a directional alignment metric generated based on the first directional probability distribution for the first protein and the second directional probability distribution for the second protein and (ii) an angular alignment metric based on the first angular probability distribution for the first protein and the second angular probability distribution for the second protein.

4. The method of claim 1, wherein determining the compression metric further comprises determining the compression metric as a function of: (i) a directional alignment metric generated based on a first directional probability distribution for the first protein and a second directional probability distribution for the second protein and (ii) an angular alignment metric based on the first angular probability distribution for the first protein and the second angular probability distribution for the second protein.

5. The method of claim 4, wherein determining the directional alignment metric further comprises determining the directional alignment metric using a metric correspond to a correct number of rotamers for the pair of proteins without the offset in accordance with a predicted local distance difference test (pLDDT).

6. The method of claim 1, further comprising determining, by the one or more processors, that the distance metric does not exceed a threshold, and
wherein selecting the therapy further comprises selecting the therapy applicable to both the first protein and the second protein of the pair of proteins, responsive to determining that the distance metric does not exceed the threshold.

7. The method of claim 1, further comprising determining, by the one or more processors, that the distance metric exceeds a threshold, and
wherein selecting the therapy further comprises selecting a subset of therapies applicable to the first protein and the second protein respectively, responsive to determining that the distance metric exceeds the threshold.

8. The method of claim 1, wherein the plurality of therapies comprises at least one of an anti-cancer drug, an anti-fungal drug, an anti-viral drug, a cardiovascular agent, a CNS agent, an antibacterial drug, an anti-inflammatory drug, or a hormonal drug.

9. The method of claim 1, wherein the plurality of therapies comprises at least one of a small molecule, an antibody, a fusion protein, a peptide, or an inhibitory nucleic acid.

10. The method of claim 1, wherein the protein structures comprise one or more of retrotransposons, oncogenes, tumor suppressors, receptors, ligands, enzymes, signaling proteins, ribosomal proteins, cytosolic proteins or transcription factors.

11. The method of claim 1, wherein the pathology is a cancer, an autoimmune disease, a metabolic disease, cardiovascular disease, a neurodegenerative disease or an infectious disease.

12. A system for selecting therapies based on structural distances among proteins encoded by open reading frames (ORFs), comprising:
one or more processors coupled with memory configured to:
obtain, for a subject suffering from or at risk of a pathology, a dataset defining a plurality of protein structures associated with at least one protein, the plurality of protein structures including a pair of proteins encoded by ORFs, the pair of proteins including a first protein and a second protein;
identify, for the pair of proteins using the dataset, (i) a first coordinate associated with the first protein and (ii) a second coordinate associated with the second protein, the second coordinate relative to the first coordinate within the plurality of protein structures;
determine an alignment metric between the pair of proteins, using an offset between the first coordinate associated with the first protein and the second coordinate associated with the second protein;
determine a compression metric based on the alignment metric and a reference alignment metric for the pair of proteins without the offset;
generate, based on the compression metric, a distance metric indicating a degree of similarity between the pair of proteins;
select, from a plurality of therapies, a therapy to address the pathology based on the distance metric; and
store, using one or more data structures, an association between the subject and the therapy.

13. The system of claim 12, wherein the one or more processors are further configured to provide, for presentation, an instruction identifying the therapy to address the pathology in the subject.

14. The system of claim 12, wherein the one or more processors are further configured to:
generate (i) a first directional probability distribution and a first angular probability distribution using the first coordinate for the first protein and (ii) a second directional probability distribution and a second angular probability distribution using the second coordinate for the second protein; and
determine (i) a directional alignment metric generated based on the first directional probability distribution for the first protein and the second directional probability distribution for the second protein and (ii) an angular alignment metric based on the first angular probability distribution for the first protein and the second angular probability distribution for the second protein

15. The system of claim 12, wherein the one or more processors are further configured to determine the compression metric as a function of: (i) a directional alignment metric generated based on a first directional probability distribution for the first protein and a second directional probability distribution for the second protein and (ii) an angular alignment metric based on the first angular probability distribution for the first protein and the second angular probability distribution for the second protein

16. The system of claim 12, wherein the one or more processors are further configured to:
determine that the distance metric does not exceed a threshold, and
select the therapy applicable to both the first protein and the second protein of the pair of proteins, responsive to determining that the distance metric does not exceed the threshold

17. The system of claim 12, wherein the one or more processors are further configured to:
determine that the distance metric exceeds a threshold, and
select a subset of therapies applicable to the first protein and the second protein respectively, responsive to determining that the distance metric exceeds the threshold

18. The system of claim 12, wherein the plurality of therapies comprises at least one of an anti-cancer drug, an anti-fungal drug, an anti-viral drug, a cardiovascular agent, a CNS agent, an antibacterial drug, an anti-inflammatory drug, or a hormonal drug.

19. The system of claim 12, wherein the plurality of therapies comprises at least one of a small molecule, an antibody, a fusion protein, a peptide, or an inhibitory nucleic acid.

20. The system of claim 12, wherein the pathology is a cancer, an autoimmune disease, a metabolic disease, cardiovascular disease, a neurodegenerative disease or an infectious disease.
